(19) United States
(12) Patent Application Publication
(10) Pub. No.: US 2013/0116510 A1
(43) Pub. Date: May 9, 2013

(54) SURGICAL ACCESS DEVICE AND SURGICAL ACCESS SYSTEM

(71) Applicant: Aesculap AG, Tuttlingen (DE)

(72) Inventors: Theodor Lutze, Balgheim (DE); Tom Schweitzer, Tuttlingen (DE)

(73) Assignee: AESCULAP AG, Tuttlingen (DE)

(21) Appl. No.: 13/670,745

(22) Filed: Nov. 7, 2012

(30) Foreign Application Priority Data
Nov. 8, 2011 (DE) 10 2011 055 129

(51) Int. Cl. A61B 1/32

(52) U.S. Cl. CPC A61B 1/32
USPC 600/208

(57) ABSTRACT

In a first aspect of the invention, a surgical access device for inserting surgical instruments into the body of a patient comprises a sleeve section defining a longitudinal axis and also a working channel. A ratio between a length and an internal diameter of the sleeve section lies in a range of approximately 4:1 to approximately 14. In a second aspect of the invention, a surgical access system comprises a surgical access device for introducing surgical instruments into the body of a patient which comprises a sleeve section defining a longitudinal axis, and also comprising an insertion instrument for inserting the access device into an abdominal wall of the patient in particular. A ratio between a length and an internal diameter of the sleeve section lies in a range of approximately 4:1 to approximately 14:1.

### CROSS-REFERENCE TO RELATED APPLICATIONS

This application claims the benefit of German application number 10 2011 055 129.8 filed Nov. 8, 2011, which is incorporated herein by reference in its entirety and for all purposes.

### FIELD OF THE INVENTION

The present invention relates to a surgical access device for introducing surgical instruments into the body of a patient, comprising a sleeve section defining a longitudinal axis and also a working channel.

Furthermore, the present invention relates to a surgical access system having a surgical access device for introducing surgical instruments into the body of a patient which comprises a sleeve section defining a longitudinal axis and also a working channel, and an insertion instrument for inserting the access device particularly into an abdominal wall of the patient.

### BACKGROUND OF THE INVENTION

Usually trocars are used for creating access to the interior of the body of a patient for minimally invasive surgical procedures. Trocars are used in particular in the field of laparoscopy and basically serve the purpose of creating a gas-tight access to the field of the operation. Trocars usually comprise a trocar sleeve and an obturator with which the trocar sleeve is closed when being introduced through the abdominal wall of the patient into the abdominal cavity for example.

A disadvantage of conventional trocars is that their trocar sleeves additionally comprise a trocar head in which there are arranged or formed a sealing unit and a valve, preferably with a standardized Luer-Lok connector, in order to fill the abdominal cavity of the patient with an inert gas. The trocar heads then usually stick out from the abdominal wall and, in the most unfavourable case, can hook themselves onto cables and hoses which are needed for the surgical procedure.

However, due to the space required by the trocar heads, it is not possible to place the trocars very close to one another. Consequently, two to three very closely adjacent points of access to the patient's body cannot be opened with trocars. At all events, this is possible with a so-called “single-port” technique which, for example, provides two to three very closely adjacent points of access in the region of the navel in a single device. Moreover, re-usable trocars in particular have a high dead weight and may be top-heavy so that there is an increased risk that they could tip over. Changing the instrument is thus made more difficult and an additional hand is needed for holding the trocar sleeve.

The aforesaid problems are circumvented particularly in the field of arthroscopy by partly dispensing completely with trocars, and endoscopic instruments are introduced directly through the tissue. Here however, the danger exists that with each change of instrument the instruments hook themselves into the layers of tissue and renewed introduction thereof is made more difficult.

An object of the present invention is to improve a surgical access device and also a surgical access system of the type initially described in such a way that a simple and safe access into the body of a patient is made possible.

### SUMMARY OF THE INVENTION

In a first aspect of the invention, a surgical access device for inserting surgical instruments into the body of a patient comprises a sleeve section defining a longitudinal axis and also a working channel. A ratio between a length and an internal diameter of the sleeve section lies in a range of approximately 4:1 to approximately 14.

In a second aspect of the invention, a surgical access system comprises a surgical access device for introducing surgical instruments into the body of a patient which comprises a sleeve section defining a longitudinal axis, and also comprising an insertion instrument for inserting the access device into an abdominal wall of the patient in particular. A ratio between a length and an internal diameter of the sleeve section lies in a range of approximately 4:1 to approximately 14:1.

### BRIEF DESCRIPTION OF THE DRAWING FIGURES

The foregoing summary and the following description may be better understood in conjunction with the drawing figures, of which:

FIG. 1: shows a partly sectional perspective view of a first exemplary embodiment of a surgical access device;

FIG. 2: a schematic longitudinal sectional view of a second exemplary embodiment of a surgical access device;

FIG. 3: a schematic longitudinal sectional view of a third exemplary embodiment of a surgical access device;

FIG. 4: a schematic illustration of a first exemplary embodiment of an insertion instrument when inserting the access device illustrated in FIG. 1;

FIG. 5: a schematic longitudinal sectional view of a second exemplary embodiment of an insertion instrument when inserting the access device illustrated schematically in FIG. 1;

FIG. 6: a schematic illustration similar to FIG. 5 wherein there are two access devices that are held one behind the other on the holding section of the insertion instrument;

FIG. 7: a schematic longitudinal sectional view of a fourth exemplary embodiment of a surgical access device;

FIG. 8A: a schematic longitudinal sectional view of a fifth exemplary embodiment of a surgical access device in the introduction position;

FIG. 8B: a side view of the surgical access device illustrated in FIG. 8A;

FIG. 8C: a view similar to FIG. 8A, wherein the access device has adopted a restraining position; and

FIG. 9: a schematic sectional view of a sixth exemplary embodiment of a surgical access device.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a surgical access device for inserting surgical instruments into the body of a patient, comprising a sleeve section defining a longitudinal axis and also a working channel, wherein a ratio between a length and an internal diameter of the sleeve section lies in a range of approximately 4:1 to approximately 14:1.

The provision of surgical access devices having sleeve sections which exhibit such a length to internal diameter relationship makes it possible to create a particularly small and compact point of access into the interior of the body of a patient. In particular, for ever decreasing instrument shafts having diameters between 3 and 5 mm, they make possible a sufficiently short overall length so that neither are there disturbing trocar heads located outside the body nor is there a risk of tipping of the access device. The proposed surgical access device can thus be formed, in particular, without trocars. Due to the resultant overall very small and compact construction of the access device, the points of access to the patient's body are also very small and non-traumatic. For example, they can be set very close to one another in the region of the navel of a patient and hence also form an alternative to the initially mentioned “single-port” technique. In any case, two or more placed access devices have the advantage that any possible deflections thereof with instruments will be less mutually interactive than is the case for trocars incorporating two or more working channels, so-called “single-port” trocars. Due to their small size, the proposed surgical access devices can be used both in the abdominal wall for laparoscopic procedures as well as in the knee for arthroscopic procedures. In particular, access devices can also be supplied in a set of different lengths so that, after firstly determining a thickness such as that of the abdominal wall for example, an operating surgeon can select the optimally matching access device which will protrude only minimally on the inside and the outside of the abdominal wall.

It is particularly expedient if the ratio between the length and the internal diameter of the sleeve section lies in a range of approximately 7:1 to approximately 11:1. Such a length to internal diameter relationship of the sleeve section, makes it possible for the surgical access device to be employed universally particularly for shaft diameters of 3 mm to 5 mm, and indeed, without the access device undesirably sticking out a long way on the inside and the outside of the abdominal wall or the knee of a patient. In other words, with the aid of the trocar-less surgical access device, it is possible to create a point of access which extends substantially just through the body tissue of the patient and is suitable for the introduction of surgical instruments. In this way in particular, the surgical access device forms a kind of plug with a passage channel which is characterised by an optimised length to diameter relationship.

Advantageously, a length of the sleeve section lies in a range of approximately 15 mm to approximately 50 mm. Preferably, a length of the sleeve section lies in a range of approximately 25 mm to approximately 40 mm. Consequently, particularly in combination with an internal diameter of the sleeve section preferably in a range of approximately 3 mm to approximately 5 mm, this results in a very small port which is sufficient to penetrate the abdominal wall in particular and to protect it from injury when introducing instruments. The application of two or more access devices in direct proximity is easily possible.

It is expedient if a first annular flange directed away from the longitudinal axis in a radial direction is formed at a proximal end of the sleeve section. This can, in particular, prevent the access device from slipping into the interior of the body of the patient through an incision in the abdominal wall or on the knee. It can be advantageous in particular, if the annular flange is opened or formed in funnel-like manner pointing in the proximal direction. The introduction of instruments into the guidance or working channel defined by the sleeve section is thereby facilitated. In particular, the annular flange may comprise a conical sliding surface which tapers in the distal direction.

It is advantageous, if the ratio of the outer diameter of the annular flange to the outer diameter of the sleeve section lies in a range of approximately 1.5:1 to approximately 3:1. Expediently, the ratio of the outer diameter of the annular flange to the outer diameter of the sleeve section lies in a range of approximately 2:1 to approximately 2.5:1. These ratios make it possible, in particular, to ensure secure placement of the first annular flange on the outside of the skin of the patient in order to prevent the access device slipping through into the interior of the body of the patient.

It is advantageous if a second annular flange directed away from the longitudinal axis in a radial direction is formed at a distal end of the sleeve section. Such a second annular flange can, in particular, also prevent the access device itself from being pulled out of an incision in the body of the patient unintentionally when pulling an instrument out of the access device.

In order not to make the introduction of the access device through an incision in an abdominal wall of the patient for example, and the withdrawal thereof after the procedure has been completed unnecessarily more difficult, it is expedient if an outer diameter of the first annular flange is greater than an outer diameter of the second annular flange.

Furthermore, it can be advantageous if a distal end of the sleeve section widens out or opens outwardly away from the longitudinal axis. Thus, in particular, projecting parts of the instrument can be fed back properly into the working channel after the withdrawal of an instrument. Furthermore, a force for retaining the access device on the patient's body can thus also be increased. Moreover, haemorrhages through the second annular flange or of the opening or widening distal end of the sleeve section can also be stopped or minimized. At the same time, a drip-edge for preventing contamination especially of an endoscope lens of an endoscope particularly in the case of haemorrhages can be formed by appropriate shaping of the distal end.

It is particularly expedient if the sleeve section is formed from a resiliently deformable material. Such an arrangement makes it possible, in particular, for the sleeve section itself to be used as a sealing element or valve because it can be squeezed together due to the tissue-tension of the tissue surrounding the sleeve section so that the working channel is sealed automatically. Furthermore, the introduction and extraction of instruments and endoscopic optics through the access device can be facilitated by forming the sleeve section from a resiliently deformable material.

It is expedient if a sealing element which projects from an inner wall of the sleeve section in the direction of the longitudinal axis is arranged or formed in the region of a proximal end of the sleeve section. Independently of whether the sleeve section is made of a resilient or inelastic material, the sealing element can seal the access device relative to an instrument shaft which can, in particular, have a somewhat smaller outer diameter than the internal diameter of the working channel. The sealing element can be formed on the sleeve section or formed in one piece manner therewith.

It is advantageous if the sealing element is arranged or formed at the level of a distal end of the first annular flange. For example, it can be formed or moulded on the sleeve section together with the first annular flange. Arranging or forming the sealing element on the sleeve section as far as possible towards the proximal end has the advantage that an instrument shaft is sealed in the desired manner practically immediately after being introduced into the access device. Thus in particular, a loss of gas through the access device can thereby be minimized.

The sealing element can be formed and produced in a particularly simply manner if it is in the form of an annular flange, a sealing bead or a sealing lip.

In order to also enable the access device to be used for laparoscopic procedures wherein the abdominal wall is raised by flooding the abdominal cavity with gas, it is expedient if a valve element is arranged or formed in the working channel. In particular, the valve element then prevents gas from escaping from the abdominal cavity when no instrument has been introduced into the working channel of the access device.

The construction and also the production of the valve element are particularly simple, if it is in the form of a duckbill or a cross slot valve. In particular, the valve element can be formed in one piece manner with the sleeve section or formed thereon by means of a moulding process for example.

It is particularly simple for the production of the access device, if the valve element is arranged or formed on the sleeve section approximately centrally between the proximal and distal ends thereof.

Furthermore it can be advantageous if the valve element is arranged or formed on the distal side of the sealing element. Preferably, it borders directly thereon. This makes it possible to introduce an instrument into the working channel which is sealed by the valve element, whereby the valve element keeps the working channel closed until such time as the sealing element can ensure a seal to the instrument shaft.

In accordance with a further preferred embodiment of the invention, provision may be made for the access device to comprise a restraining device for preventing a movement of an access device in the proximal direction particularly one that has been inserted into an abdominal wall of a patient. Self evidently, the restraining device can also prevent or make more difficult a movement out of the body of a patient at some other position. The effect of the restraining device is desirable and advantageous particularly when an instrument is being withdrawn from the working channel of the access device.

The restraining device can be formed in a particularly simple manner if it comprises at least one restraining member which comprises a restraining surface facing in the proximal direction. In particular, the restraining surface can extend transversely relative to a longitudinal axis of the working channel so that, upon the introduction of the sleeve section into the body tissue of the patient, the at least one restraining member will hook itself into the surrounding body tissue by a movement in the proximal direction and can thereby prevent unintentional withdrawal of the access device or at least make it significantly more difficult.

It is particularly advantageous if two, three or more restraining members are provided. In particular, the restraining members can all be formed identically or may differ in type, size and construction.

In particular, a particularly simple construction of the access device can be achieved in that the at least one restraining member is in the form of a projection from the sleeve section which is directed away from the longitudinal axis in a radial direction.

The access device can be introduced into the body of the patient in the distal direction in a particularly simple manner if the projection comprises a sliding surface which is directed away from the longitudinal axis and is inclined in the distal direction.

The at least one restraining member can exercise its restraining function to particularly good effect, if the projection extends at least partly over the periphery of the sleeve section in the peripheral direction. Preferably, the projection extends over the entire periphery of the sleeve section. In other words, the projection can also be formed so as to surround the sleeve section in ring-like manner.

It is advantageous if the at least one restraining member is held on the sleeve section in moveable manner and is moveable from an introduction position into a restraining position. Due to this arrangement, it is possible to introduce the access device into the body tissue in a simple manner when the at least one restraining member adopts the introduction position, and to hold it on the body of the patient in a defined and secure manner when the at least one restraining member adopts the restraining position.

It is expedient if the at least one restraining member is mounted on the sleeve section such as to be pivotal about a pivotal axis. Such an arrangement can be formed in a simple manner and prescribe a defined movement of the at least one restraining member.

It is advantageous if the pivotal axis extends transversely or substantially transversely with respect to the longitudinal axis. This permits the at least one restraining member to pivot in such a way that it projects laterally from the sleeve section in the restraining position for example.

In order to simplify the arrangement particularly in the case of very small access devices, it is advantageous if the at least one restraining member is mounted on the sleeve section in moveable manner by means of a pivotal joint or a hinge joint and in particular, the hinge joint can be in the form of a film hinge so that the joint can be formed in one piece manner with the sleeve section.

In order for the restraining function exercised by the restraining device to be as uniform as possible, it is expedient for two restraining members to be arranged or formed diametrically opposite each other taken with respect to the longitudinal axis.

The construction of the access device can be simplified further if the at least one restraining member is formed such as to be tab-shaped. In particular, tab-shaped can mean that the support element is in the form of a flat elongated cuboid.

The access device can be produced in a simple manner and be anchored in the body tissue like a dowel, if the distal end of the at least one restraining member is arranged or formed on the sleeve section and if a free proximal end of the at least one restraining member is directed somewhat away from the longitudinal axis in the proximal direction. The at least one restraining member can thus be in the form of a sort of barb.

Furthermore, it can be advantageous if the at least one restraining member forms a section of the wall of the sleeve. In particular, it can thus form a part of the sleeve-wall of the sleeve section in the introduction position.

It is expedient if the at least one restraining member forms a part of a sleeve wall of the sleeve section in the introduction position and if it protrudes or is deflected at least partly away from the longitudinal axis in a radial direction in the restraining position. In particular, the access device can then be transferred from the introduction position into the restraining position in a simple and certain manner, by deflecting or pivoting the at least one restraining section for example.

In order to enable particularly secure hooking or fixing of the access device in the body tissue of the patient to be achieved, it is expedient if the at least one restraining member is arranged or formed in the region of the distal half of the sleeve section. In this way, when withdrawing the access device in the proximal direction, more body tissue has to be negotiated than in the case of an arrangement of the at least one restraining member in the region of a proximal end of the sleeve section.

In accordance with a preferred embodiment of the invention, provision may be made for the restraining surface to define a plane which includes an angle of between approximately 75° and 105° with the longitudinal axis. Preferably, the angle amounts to approximately 90°. If the included angle is an acute angle which points in the proximal direction, then a barbed construction of the restraining device can be formed in a simple manner.

Furthermore, it can be expedient if the access device comprises at least one restraining member in the form of an external thread which is arranged or formed on the sleeve section. The access device can then be quasi screwed or rotated into the body tissue for example. The flanks of the screw thread then prevent a movement of the sleeve section in the proximal direction in a simple manner so that corresponding forces have to be applied for removing it during the withdrawal process.

It is advantageous if at least the sleeve section is made of an elastomer, preferably from a thermoplastic elastomer or an elastomer foam. Thus for example, as already described hereinabove, the sleeve section can also serve as a sealing element or valve element for the access device itself if, in particular, it is formed to be so resilient that it can be squeezed together by the body tissue surrounding it in order to close the working channel. Furthermore for example, the sleeve section can be made of an elastomer, optionally provided annular flanges at the proximal and/or distal end of the sleeve section can be made of a substantially inelastic synthetic material. In dependence on the function of the corresponding parts or elements of the access device, they can be formed as appropriate from a resilient or from a substantially inelastic material. The production of the access device from one or more synthetic materials makes for a simple and economical production process, by injection moulding for example.

It is expedient if the sleeve section formed from the elastomer itself forms the valve element. A valve element which is complicated to arrange and form in the working channel is then no longer necessary in this case.

The production of the access device can be simplified further, if it is made entirely of an elastomer. Preferably, it is made of a thermoplastic elastomer or an elastomer foam.

In order to simplify the introduction of the access device into the body of the patient and/or the introduction of an instrument through the access device, it is expedient if an inner surface and/or an outer surface of the access device is provided with a friction-reducing coating.

Furthermore, the stability of the access device can be increased and also the production thereof can be simplified if it is formed in one piece manner.

Furthermore, the present invention relates to a surgical access system comprising a surgical access device for introducing surgical instruments into the body of a patient which comprises a sleeve section defining a longitudinal axis, and also comprising an insertion instrument for inserting the access device into an abdominal wall of the patient in particular, wherein a ratio between a length and an internal diameter of the sleeve section lies in a range of approximately 4:1 to approximately 14:1. As has already been explained in detail hereinabove, such an access system incorporating the improved surgical access device makes it possible to create one or more access points into the body of a patient in a simple manner. In particular, one can thereby dispense with large trocars having trocar heads and the initially described disadvantages.

It is advantageous if the surgical access system comprises one of the access devices described above. The surgical access system then likewise exhibits the advantages described above in connection with preferred embodiments of the surgical access devices.

In order to simplify the insertion of the surgical access device, it is advantageous if the insertion instrument comprises a holding section for at least one surgical access device. Preferably, the holding section can be formed in such a manner that two, three or even more access devices can be held thereon simultaneously. This permits one, two or yet more access points or ports into the interior of the body of the patient to be created successively using just one insertion instrument.

In order to prevent a surgical access device that is held on the holding section from being able to slip down off the holding section in the distal direction when this is not desired, it is expedient if a stop acting in the proximal direction is arranged or formed at the distal side of the holding section.

Such a stop can be formed in a simple manner if it has a larger outer diameter than the holding section. The stop can, in particular, be in the form of an annular projection or flange which, however, preferably does not have sharp edges. This enables the sleeve section of the access device to be pushed quite deliberately over the stop whereby the sleeve section can preferably be temporarily expanded for this purpose.

It is advantageous for the holding section to be of a length which corresponds to at least twice the length of the sleeve section of a surgical access device. In this way, at least two or even more surgical access devices can be held on the holding section in dependence on how long the holding section actually is.

In order to place the at least one access device which is held on the holding section in the body tissue of the patient in the desired manner, it is advantageous if a feed element having a stop surface facing in the distal direction is arranged or formed on the proximal side of the holding section. Such a stop prevents the sleeve section that is held on the holding section from being arbitrarily displaced in the proximal direction duannular the introduction thereof. The holding section with such a stop makes it possible to introduce and place the access device in the body of the patient in a safe and purposeful manner.

It is advantageous if the feed element is held immovably on the holding section. In particular in such a case, one can completely dispense with a stop adjoining the retaining section at the distal side thereof. For example, the sleeve section can be securely held on the holding section in a simple manner in that the internal diameter of the sleeve section is a little smaller than the outer diameter of the holding section. In particular, in combination with a restraining device, it is practically impossible to unintentionally pull a sleeve section, which has been placed by the insertion instrument, out of the body tissue together with the insertion instrument when the insertion instrument is being withdrawn in the proximal direction.

Alternatively, it can also be expedient if the feed element is formed such as to be moveable relative to the holding section in the distal and the proximal direction. In particular, one or more access devices can be successively pushed down off the holding section in the distal direction in this way.

The insertion instrument can be formed in a simple manner if the feed element is in the form of a sleeve surrounding the holding section. Consequently, the distal end of the sleeve can form a stop surface acting in the distal direction upon which the proximal end of the sleeve section can be supported duannular insertion into the body of the patient.

The handling of the insertion instrument can be improved in a simple manner if a proximal end thereof is in the form of a handle element or carries a handle element.

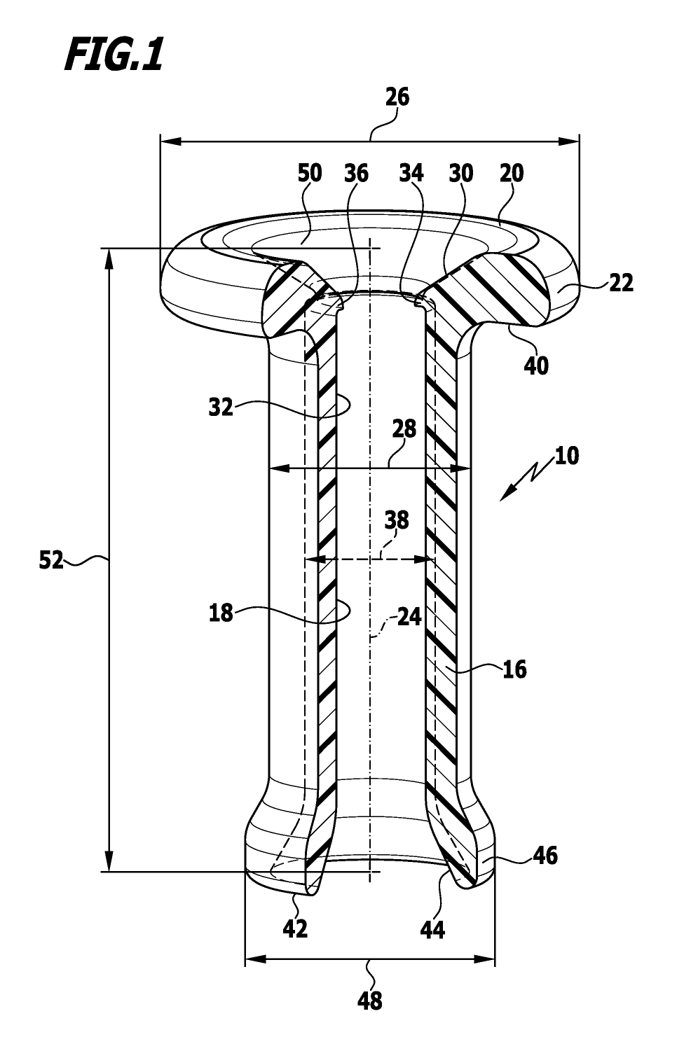

A surgical access device 10 designated as a whole by the reference symbol 10 is illustrated schematically in FIG. 1. It forms a part of a surgical access system designated as a whole by the reference symbol 12 which, furthermore, comprises an insertion instrument 14.

The access device 10, which may also be referred to as a “mini-port”, comprises a hollow cylindrical sleeve section 16 which defines a working channel 18 in the interior thereof. A proximal end 20 of the sleeve section 16 is provided with a first annular flange 22 which extends away from a longitudinal axis 24 defined by the sleeve section 16 in a radial direction. The ratio of the outer diameter 26 of the first annular flange 22 to the outer diameter 28 of the sleeve section 16 preferably lies in a range of approximately 2:1 to approximately 2.5:1.

The working channel 18 widens out conically in the proximal direction in the region of the first annular flange 22 thereby forming an inclined, funnel-like annular surface 30, the surface-normal of which is inclined to the longitudinal axis 24 in the proximal direction. In the transition region between the annular surface 30 and a hollow cylindrical inner surface 32 of the working channel 18, there is formed an annular, at least somewhat resilient sealing element 34 in the form of an annular flange 36. Alternatively, the sealing element 34 can also be in the form of a sealing bead or a sealing lip. Consequently, the internal diameter 38 of the working channel 18 is somewhat reduced in the region of the sealing element 34.

The first annular flange 22 has an annular surface 40 which extends substantially transversely with respect to the longitudinal axis 24 and faces in the distal direction and which also defines a stop surface for body tissue. On the distal side thereof, the internal diameter of the sleeve section 16 widens out conically towards the distal end 42 thereof whereby the thus formed annular surface 44 is inclined to the longitudinal axis 24 in the distal direction. An opening angle defined by the annular surface 44 is somewhat less than half as large as an opening angle defined by the annular surface 30. The end 42 is defined by a second annular flange 46, the outer diameter 48 of which is significantly smaller than the outer diameter 26.

Both of the annular flanges 26 and 46 are completely rounded and have no sharp edges. The annular surface 30 of the first annular flange 22 forms a bell mouth 50 which serves as a means for assisting the introduction of surgical instruments into the working channel 18 and therethrough into the body of a patient.

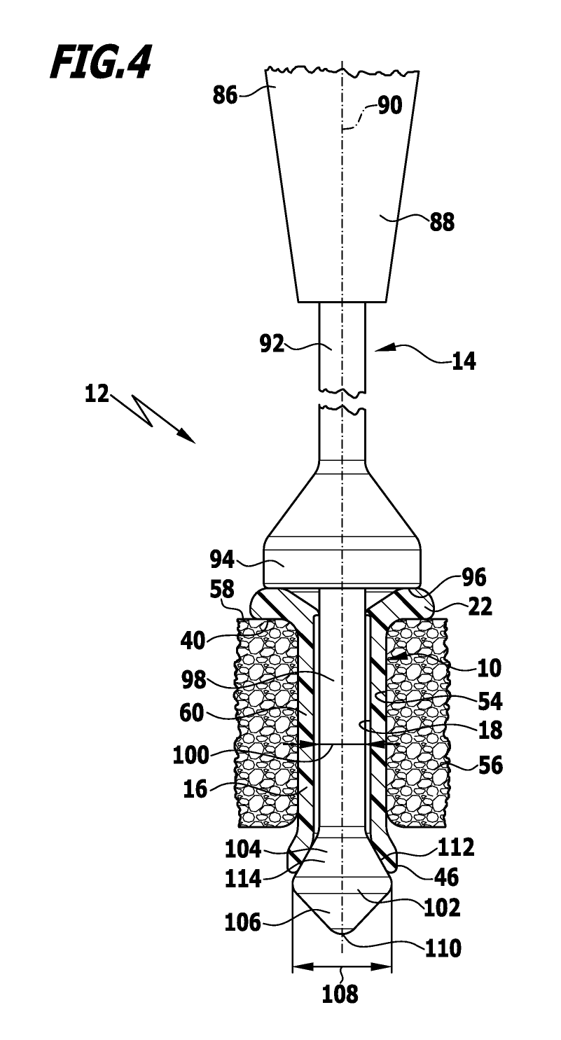

The overall length 52 of the sleeve section 16 between the ends 20 and 42 has a characteristic relationship to the internal diameter 38 of the sleeve section. In the exemplary embodiment of an access device 10 illustrated schematically in FIG. 1, it lies within a range of approximately 5:1 to approximately 7:1. This relationship could however lie within a range of approximately 4:1 to approximately 8:1. In order to let the size of the access device 10 become somewhat clearer, values in a range of approximately 15 mm to approximately 50 mm can be attributed to the overall length 52. For a large number of patients, it might be sufficient to provide for the access device 10 to have an overall length 52 of approximately 25 mm to approximately 40 mm. As illustrated schematically in FIG. 4, this makes it possible for the access device 10 to be inserted into a small incision 54 in an abdominal wall 56 of a patient by means of the insertion instrument 14 so that the annular surface 40 can abut or, better expressed, come into abutment on an outer surface 58 of the abdominal wall 56 in order to prevent the access device 10 from being introduced any further into the body of the patient. In a similar way, the second annular flange 46 serves to prevent the access device 10 from being pulled out of the incision 54 inadvertently.

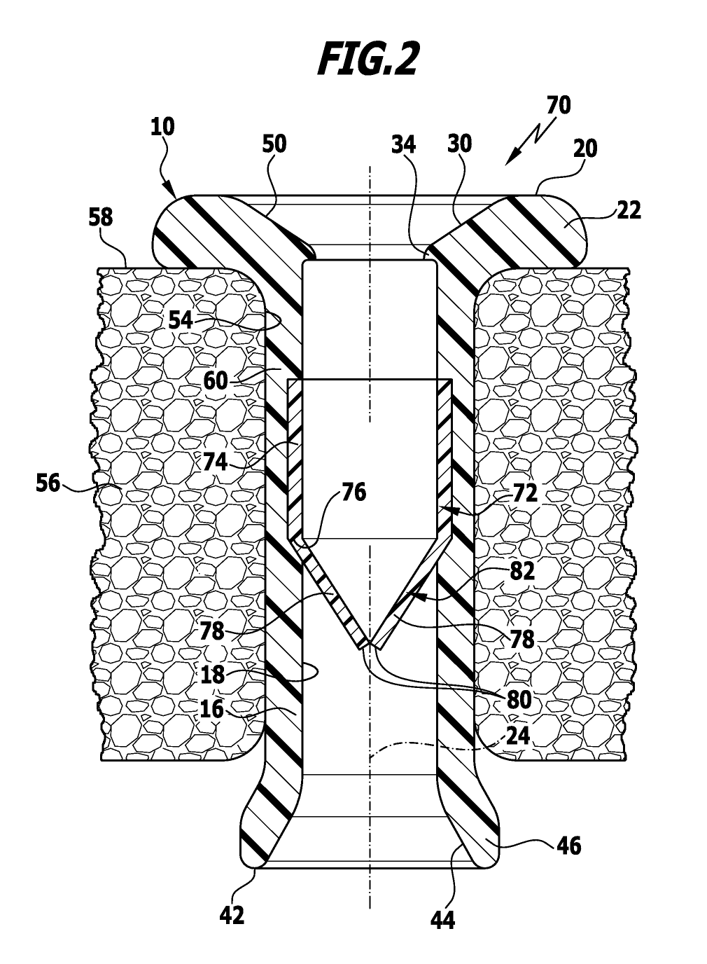

A second exemplary embodiment of a surgical access device is schematically illustrated in FIG. 2 and is designated as a whole by the reference symbol 70 therein. The basic construction thereof corresponds to that of the access device 10 and differs therefrom only in that it comprises a valve element 72 which is arranged in the working channel 18. The valve element 72 comprises a short sleeve section 74 which is arranged approximately centrally in the working channel 18 between the ends 20 and 42 in such a manner that the sleeve section 74 forms a part of the wall 60 and an inner wall surface of the sleeve section 74 forms a part of an inner wall surface of the sleeve section 16. Two valve flaps 78 that are inclined at an angle to the longitudinal axis 24 and point in the distal direction project from an annular end face 76 of the sleeve section 74 facing in the distal direction, the free ends 80 of said valve flaps abutting each other and closing the working channel 18 in gas-tight manner. The valve element 72 is thus in the form of a duckbill valve 82 but could also be in the form of a cross slot valve or some other similar type of valve in order to close the working channel 18 in gas-tight manner. Moreover, the valve element 72 could also be arranged further towards the proximal end such as directly adjacent the sealing element 34 for example.

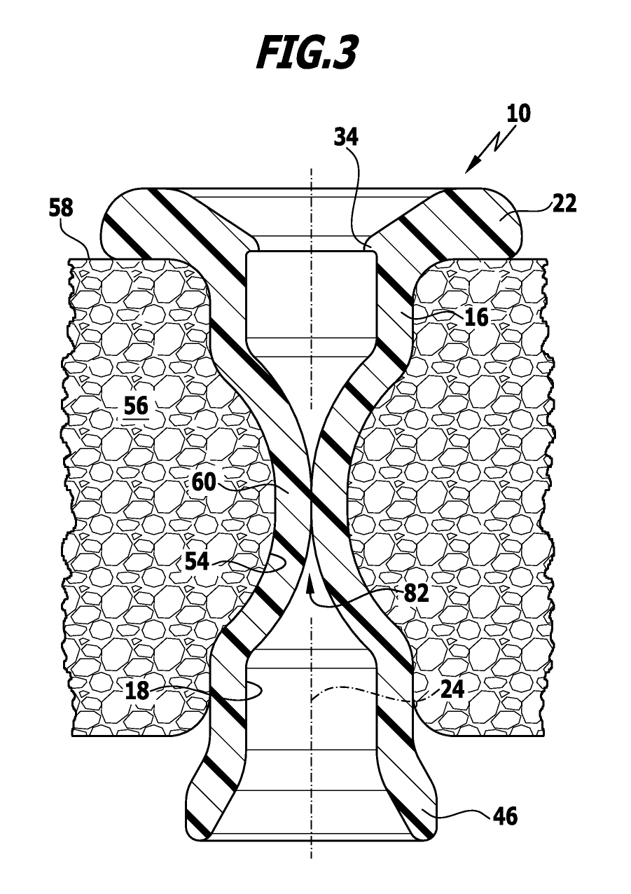

Other than is the case for the access device 70 which is schematically illustrated in FIG. 2, one can dispense with an additional valve element 72 in the case of the access device 10 if, for example, a wall 60 bounding the working channel 18 is made from a resilient material such as an elastomer for example, and in particular a thermoplastic elastomer or an elastomer foam, so that a tissue-tension of the tissue of the abdominal wall 16 surrounding the sleeve section 16 is of itself sufficient to squeeze the sleeve section 16 together in such a way that the working channel 18 is closed as is illustrated schematically in FIG. 3. Due to this special arrangement, the sleeve section comprising a resilient wall 60 itself forms the valve element 72. For the purposes of opening the latter after the introduction of an instrument, the wall 60 is pressed outwardly on all-sides as would similarly be the case too for the duckbill valve 82 or any other type of valve element.

The insertion instrument 14, which is illustrated schematically in FIGS. 4 to 6, serves for the insertion of the access devices 10 and 70 and also further access devices which will be described in more detail hereinafter. It comprises a handle element 88 at the proximal end 86 with the aid of which an operating surgeon can grasp, hold and guide the insertion instrument 14. The insertion instrument 14 can, in particular, be formed such that it is rotationally symmetrical about a longitudinal axis 90 defined thereby.

On the distal side of the handle element 88, there extends a shaft section 92 which can have a specific length in dependence on the purpose for which the insertion instrument 14 is to be used. The outer diameter of the end of the shaft section 92 widens out and thus forms a conical feed element 94 which comprises a stop surface 96 facing in the distal direction. Protruding from the stop surface 96 there is a cylindrical holding section 98 having an outer diameter 100 which corresponds approximately to the outer diameter of the shaft section 92. The outer diameter 100 is smaller than the internal diameter 38, but is somewhat larger than the internal diameter defined by the sealing element 34. This makes it possible for an access device 10 to be pushed onto the holding section 98 by its proximal end 20. Due to the described ratios of the diameters, the access device 10 is held on the holding section 98 by virtue of the frictional forces effective between the latter and the sealing element 34.

Adjoining the holding section 98 at the distal end thereof, there is a spreading body 102 which is formed from a cone 104 and a tapered-pin 106. The surface areas of the cone 104 and the tapered-pin 106 are connected to one another quasi directly and define a maximum outer diameter 108 of the spreading body 102 which is just a little smaller than the outer diameter 48 of the second annular flange 46. Commencing from the holding section 98, there is firstly formed the cone 104 which has an outer diameter that increases up to the maximum outer diameter 108. Following the cone 104 on the distal side thereof, is the tapered-pin 106 which has a rounded off peak 110 pointing in the distal direction. The transition region between the cone 104 and the tapered-pin 106 is also completely rounded. An outer wall surface 112 of the cone 104 serves as a contact surface or stop surface for the annular surface 44. The cone 104 of the spreading body 102 thus forms a stop 114 which acts in the proximal direction and prevents the access device 10 that has been pushed onto the holding section 98 from slipping down off the holding section 98 in the distal direction.

For the purposes of inserting the access device 10, the latter is pushed over the spreading body 102 onto the holding section 98. This is easily possible if it is made of a resiliently deformable material. The access device 10 is pushed onto the holding section 98 until the first annular flange 22 rests against the stop surface 96. After opening the abdominal wall 56, an operating surgeon can then push the insertion instrument 14 with the spreading body 102 forwardly into the incision 54 until the annular surface 40 comes to rest on the outer surface 58 of the abdominal wall 56. The access device 10 to be utilised is preferably selected from an available set of access devices having different overall lengths in such a way that, due to the selected overall length 52, the second annular flange 46 protrudes out somewhat from the incision 54 inside the patient.

When the access device 10 has been placed in the desired way, the insertion instrument 14 can be pulled out of the working channel 18 in the proximal direction. To this end, the access device 10 is held at the first annular flange 22 and the insertion instrument 14 is pulled out. This method of procedure is utilised when the feed element 94 and the holding section 98 are arranged or formed such that they are immoveable relative to each other.

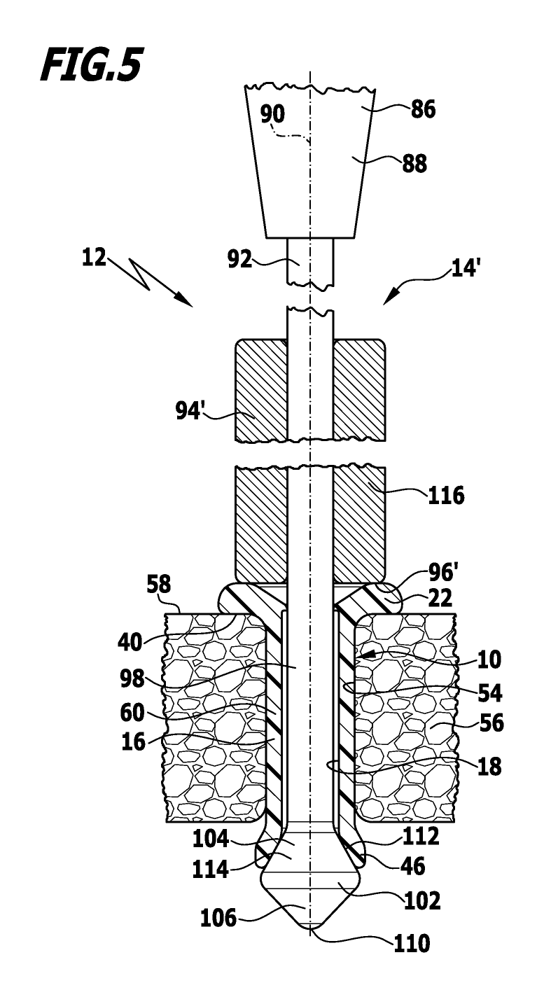

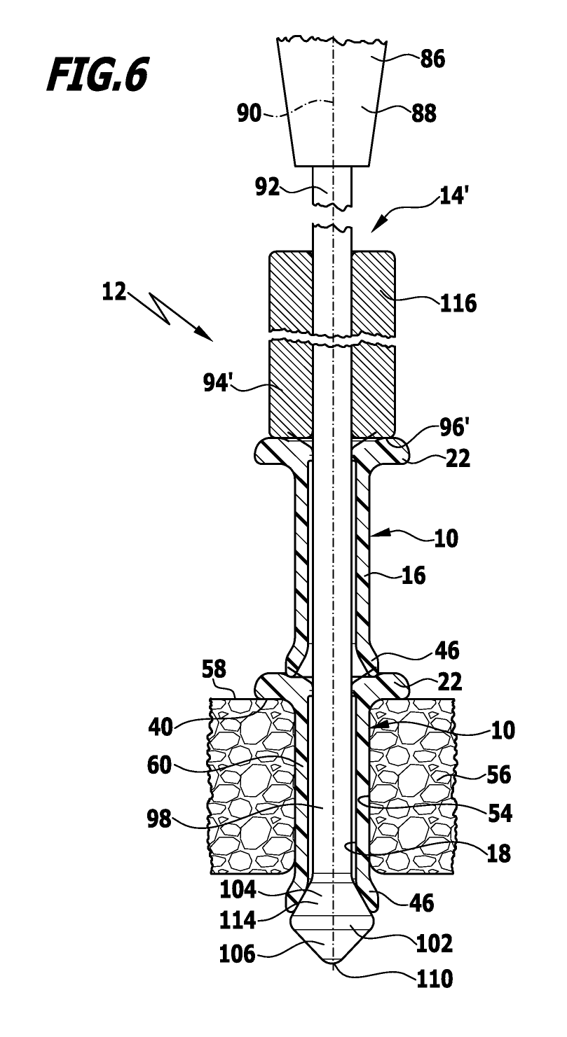

A slightly modified insertion instrument 14′ which comprises a feed element 94′ in the form of a sleeve 116 that is held in displaceable manner on the holding section 98 is illustrated schematically in FIGS. 5 and 6. This has a stop surface 96′ facing in the distal direction, from which projects the holding section 98 likewise oriented in the distal direction.

For the purposes of applying the access device 10, the access device 10 is pushed forwardly by its proximal end 20 over the spreading body 102 and onto the holding section 98 from the distal end in like manner to the insertion instrument 14. The insertion instrument 14′ is introduced into the incision 54 in the abdominal wall with the spreading body 102 in front. In order to prevent the access device 10 on the holding section 98 from being pushed back in the proximal direction, the operating surgeon holds the feed element 94′ in the position illustrated schematically in FIG. 5 so that the second annular flange 46 of the access device 10 rests on the cone 104 on the one hand and the first annular flange 22 thereof rests against the stop surface 96 on the other. Withdrawal of the insertion instrument 14 is simplified somewhat by the feed element 94′. The operating surgeon holds the feed element 94′ in position and pulls the holding section 98 out of the working channel 18 in the proximal direction by grasping the handle element 88. The feed element 94′ serves as a sort of hold-down member and remains in position until the spreading body 102 impinges on the feed element 94′. The retaining forces exerted by the abdominal wall 56 on the access device 10 are then sufficient to pull the spreading body 102 through the first annular flange 22.

In dependence on the length of the holding section 98, it is also possible to hold two or more access devices 10 on the holding section 98 one behind the other and to apply them successively in the manner described with the aid of the insertion instrument 14′ as is illustrated schematically in FIG. 6. The feed element 94′ then forms a stop for the most proximal of the access devices 10 that have been pushed onto the holding section 98. This in turn forms a stop for the next access device 10 that has been pushed on at the distal side thereof.

In consequence, a first access device 10 can be applied into an incision 54 in the abdominal wall 56 with the aid of the insertion instrument 14′. The holding section 98 is withdrawn relative to the feed element 94′ until the spreading body 102 comes to rest on the second annular flange 46 of the following access device 10 that is held on the holding section 98. The one or more access devices 10 remaining on the holding section 98 can then either be applied as illustrated and described in principle in FIG. 6 or as schematically illustrated and described in FIG. 5. For a simplified application of the access devices 10, the insertion instrument 14′ can be further optimised, for example, in the manner of a “Multifire Applicator” in which the feed element can be moved backwards and forwards in a desired manner with the aid of an actuator element that is arranged on the handle element and coupled to the feed element 94′ so that an operating surgeon does not have to hold the feed element 94′ with his hand.

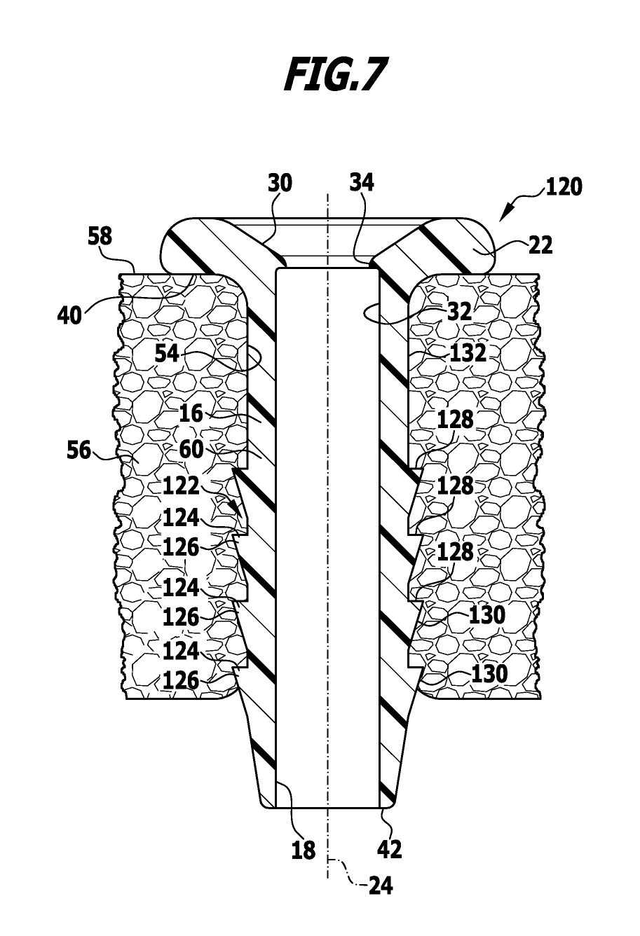

Both the access device 10 and the access device 70 may optionally comprise a restraining device for preventing an access device that has been inserted into the abdominal wall 56 from moving in the proximal direction. A further exemplary embodiment of an access device is illustrated in exemplary manner and provided with the reference symbol 120 in FIG. 7. It differs, inter alia, from the access device 10 in that the sleeve section 16 does not comprise a second annular flange, but rather the working channel 18 extends up to the distal end 42 of the access device 120 along a straight line.

Furthermore, the access device 120 comprises a restraining device 122 incorporating at least one restraining member 124. Four practically identical restraining members 124 are illustrated exemplarily in FIG. 7. They are each formed by a projection 126 which projects away from the longitudinal axis 24 of the sleeve section 16 in the radial direction and comprises a restraining surface 128 facing in the proximal direction. Furthermore, each projection 126 comprises a sliding surface 130 which faces away from the longitudinal axis 24 and is inclined in the distal direction. The projection 126 may extend only over a part of the periphery of the sleeve section 16, optionally, it extends over the entire periphery of the sleeve section 16 and is consequently in the form of an annular projection. The restraining surfaces 128 and sliding surfaces 130 described above create a kind of saw tooth profile in the sectional view in FIG. 7, one could also say that an outer contour of the access device 120 is undercut in the form of a Christmas tree. The sliding surfaces 130 can extend transversely with respect to the longitudinal axis 24, but they could be inclined relative thereto, namely, in such a manner that an undercut is preferably defined between the restraining surface 128 and an outer surface 132 of the sleeve section.

If the access device 120 is inserted into the incision 54 with the aid of one of the insertion instruments 14 or 14′, the restraining members 124 lead to it becoming hooked into the body tissue when the sleeve section 16 is pulled in the proximal direction. Inadvertent pulling of the access device 120 out of the incision 54 can be prevented or at least the risk thereof is diminished in this way. As was the case for the access devices 10 and 70, slippage of the access device 120 through the abdominal wall 56 is prevented by the first annular flange 22.

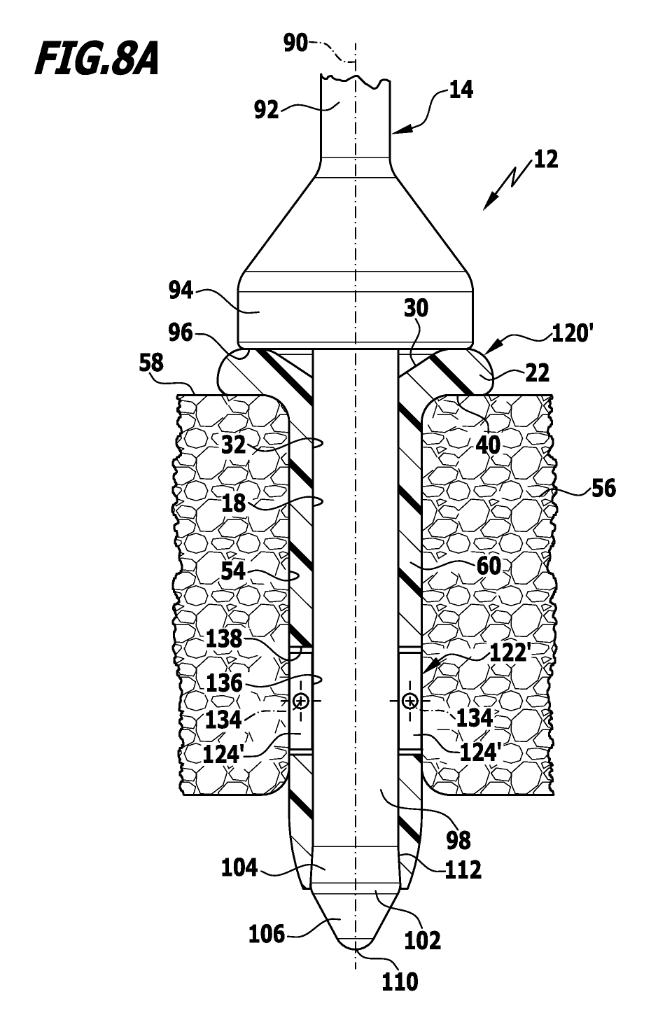

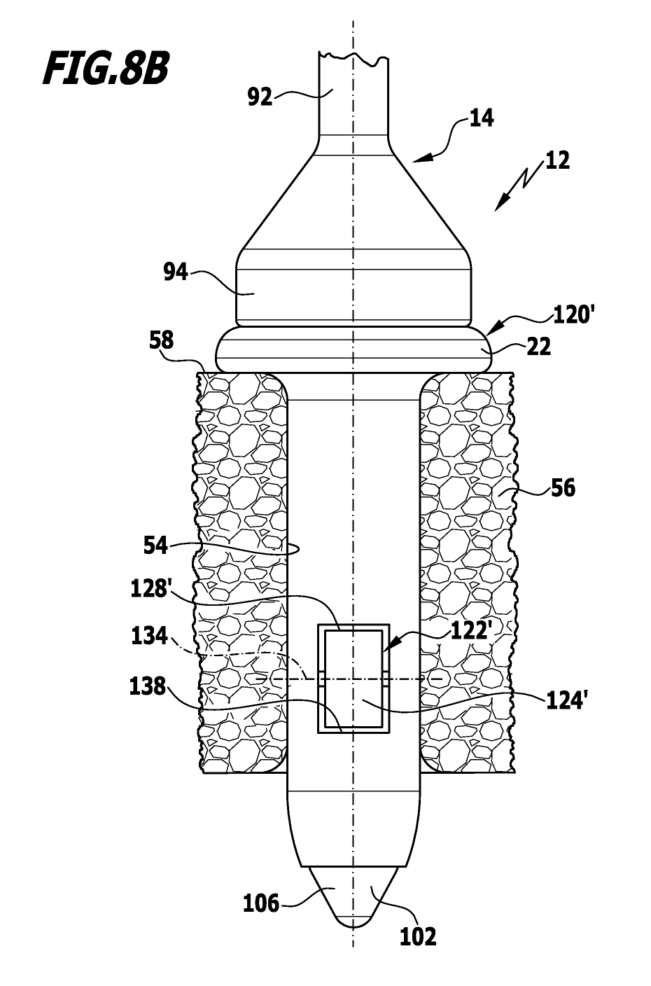

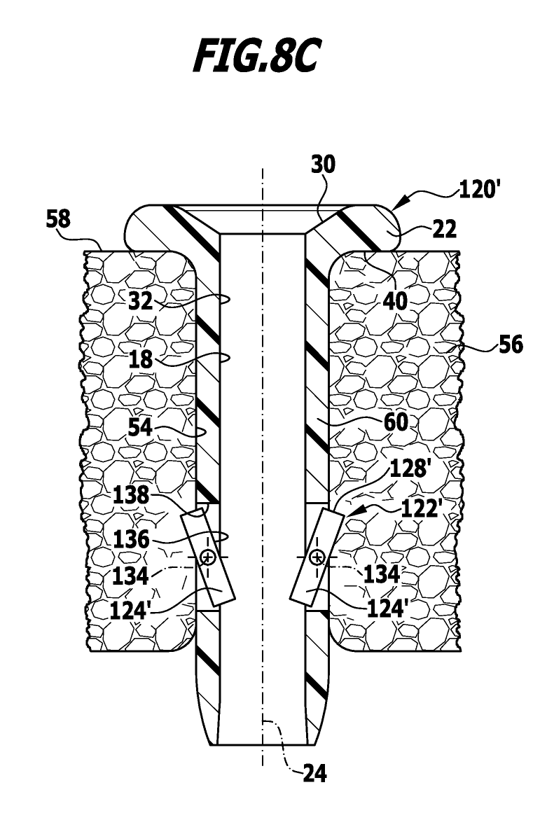

A further exemplary embodiment of a surgical access device is illustrated schematically in FIGS. 8A to 8C and is designated as a whole by the reference symbol 120′ therein. It differs from the access device 120 merely by the design of the restraining device 122′. This comprises two substantially plate like restraining members 124′ which form a part of the wall 60 of the sleeve section 16. They are mounted in moveable manner on the sleeve section 16, namely, they are pivotal about a pivotal axis 134 which is oriented transversely with respect to the longitudinal axis 24. The pivotal axis 134 runs substantially through the centre of gravity of the restraining members 124′. The latter adopt the introduction position in FIGS. 8A and 8B, i.e. they form a part of the wall 60 of the sleeve section 16.

If the proximal end of the restraining member 124′ is swung outwardly as is exemplarily illustrated in FIG. 8C, the restraining member 124′ adopts the restraining position. A rear end face of the restraining members 124′ facing in the proximal direction thus forms a restraining surface 128′. This may, in particular, include an angle of between 0° and 90° with the longitudinal axis 24 in dependence on the pivotal angle. An inner surface 136 of the restraining member 124′ can likewise form a restraining surface, namely, in the case where the restraining member 124′ is swung through approximately 90° so that the inner surface 136 outside the sleeve section 16 bites into the body tissue and a movement of the access device 120′ in the proximal direction is prevented or at least made more difficult.

The insertion instruments 14 or 14′ can be used for the purposes of applying the access device 120′. Optionally, it is also conceivable in essence to dispense with the cone 104 in the insertion instruments 14 and 14′ so that only the tapered-pin 106 having a rounded-off peak 110 pointing in the distal direction remains. For the application of the access device 120′, the holding section 98 is pushed into the working channel 18 in the way that has already been described. The holding section 98 thereby blocks any movement of the restraining members 124. These thus form a part of the wall 60 as they substantially fill-up completely a window-like through-opening 138 that is formed therein. When the insertion instrument is withdrawn, the restraining members 124′ can be swung about the pivotal axis 134 in the previously described manner so that the access device 120′ is secured in the body tissue in the desired manner.

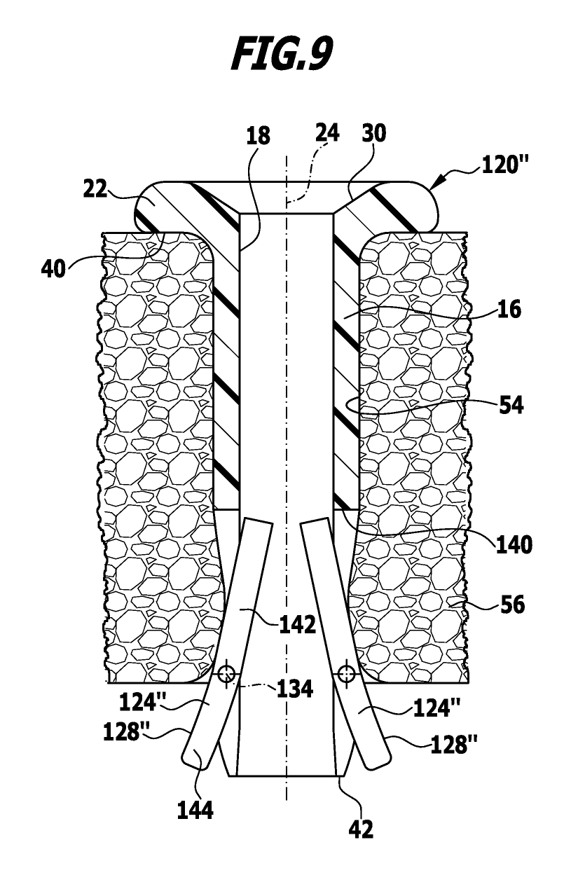

A further exemplary embodiment of an access device designated as a whole by the reference symbol 120″ is illustrated schematically in FIG. 9. The construction thereof is similar to that of the access device 120′ and differs therefrom only in regard to the size and position of the restraining members 124″. The restraining members 124″ are arranged diametrically opposite each other taken with respect to the longitudinal axis 24 and they are disposed in an indentation 140 which, outgoing from the end 42, extends in the proximal direction over approximately 40% of the overall length 52 of the sleeve section 16. They are each mounted such as to be pivotal about a pivotal axis 134 which runs transversely with respect to the longitudinal axis 24 but does not however intersect it. In addition, the pivotal axis 134 does not run through the centre of gravity of the restraining members 124″. The centre of gravity is located somewhat on the proximal-side of the pivotal axis 134 so that the surrounding body tissue presses the lever section 142, which extends in the proximal direction starting from the pivotal axis 134, in the direction of the longitudinal axis 24 and a lever section 144 of the restraining members 124″, which extends in the distal direction starting from the pivotal axis 134, can swing laterally outwards. In the swung-out position thereof which corresponds to the restraining position of the access device 120′, the swung-out lever sections 144 take on the function of the second annular flange 46 in the access device 10. The only difference is that the lever sections 144 are merely swung out on a temporary basis and do not extend over the entire periphery of the sleeve section 16.

For the purposes of inserting the access device 120″, the same insertion instrument can be used as was employed for the insertion of the access device 120′. In the case of the access device 120″ too, the restraining members 124 are forced into the introduction position when the holding section 98 passes through the working channel 18. When the insertion instrument is removed from the access device 120″, the body tissue forces the restraining members 124″ into the restraining position described above which is illustrated schematically in exemplary fashion in FIG. 9. Restraining surfaces 128″ of the lever sections 144 facing at least partly in the proximal direction then form a stop which acts in the proximal direction.

The access devices 120′ and 120″ are preferably formed from an inelastic synthetic material and thereby exhibit increased stability. This is expedient, since the pivotal restraining members 124′ and 124″ can then swing-out in a defined manner and anchor themselves in the body tissue.

The access device 10, 70 and 120 can, in particular, be made of an at least partially resilient material. The first annular flange 22 and the second annular flange 46 can be formed in one piece manner with the sleeve section 16 or be formed thereon by means of a moulding process for example. It is conceivable in particular for the restraining device 122 of the access device 120 to be formed in one piece manner with the sleeve section 16 or, should another material be used therefor, by forming it thereon by means of a moulding process. In order to optimise the process of hooking into body tissue, the restraining members 124 can be formed from a synthetic material which is less resilient than the sleeve section 16 for example.

Optionally, all of the access devices described above can be provided with a friction-reducing coating, namely, on both the inner surfaces 32 and on the outer surfaces of the sleeve section. Consequently, both the process of introducing the access devices into the body and also the introduction of instruments into the working channel can be facilitated thereby.

1. A surgical access device for inserting surgical instruments into the body of a patient, comprising a sleeve section defining a longitudinal axis and also a working channel, wherein a ratio between a length and an internal diameter of the sleeve section lies in a range of approximately 4:1 to approximately 14:1.

2. A surgical access device in accordance with claim 1, wherein the ratio between the length and the internal diameter of the sleeve section lies in a range of approximately 7:1 to approximately 11:1.

3. A surgical access device in accordance with claim 1, wherein a length of the sleeve section lies in a range of approximately 15 mm to approximately 50 mm, preferably within a range of approximately 25 mm to approximately 40 mm.

4. A surgical access device in accordance with claim 1, wherein a first annular flange directed away from the longitudinal axis in a radial direction is formed at a proximal end of the sleeve section.

5. A surgical access device in accordance with claim 4, wherein a ratio of the outer diameter of the first annular flange and the outer diameter of the sleeve section lies in a range of approximately 1.5:1 to approximately 3:1, preferably in a range of approximately 2:1 to approximately 2.5:1.

6. A surgical access device in accordance with claim 1, wherein a second annular flange directed away from the longitudinal axis in a radial direction is formed at a distal end of the sleeve section.

7. A surgical access device in accordance with claim 1, wherein a distal end of the sleeve section widens out or opens outwardly away from the longitudinal axis.

8. A surgical access device in accordance with claim 1, wherein the sleeve section is formed from a resiliently deformable material.

9. A surgical access device in accordance with claim 1, wherein a sealing element projecting from an inner wall of the sleeve section in the direction of the longitudinal axis is arranged or formed in the region of a proximal end of the sleeve section.

10. A surgical access device in accordance with claim 9, wherein the sealing element is in the form of an annular flange, a sealing bead or a sealing lip.

11. A surgical access device in accordance with claim 1, wherein a valve element is arranged or formed in the working channel.

12. A surgical access device in accordance with claim 11, wherein the valve element is in the form of a duckbill or a cross slot valve.

13. A surgical access device in accordance with claim 12, wherein the valve element is arranged or formed on the sleeve section approximately centrally between its proximal and distal end (20, 42).

14. A surgical access device in accordance with claim 1, characterized by a restraining device for preventing a movement of an access device in the proximal direction particularly one that has been inserted into an abdominal wall of a patient.

15. A surgical access device in accordance with claim 14, wherein the restraining device comprises at least one restraining member which comprises a restraining surface facing in the proximal direction.

16. A surgical access device in accordance with claim 15, wherein the at least one restraining member is held on the sleeve section in moveable manner and is moveable from an introduction position into a restraining position.

17. A surgical access device in accordance with claim 15, wherein the distal end of the at least one restraining member is arranged or formed on the sleeve section and wherein a free proximal end of the at least one restraining member is directed somewhat away from the longitudinal axis in the proximal direction.

18. A surgical access device in accordance with claim 1, wherein at least the sleeve section is made of an elastomer, preferably from a thermoplastic elastomer or an elastomer foam.

19. A surgical access device in accordance with claim 18, wherein the sleeve section formed from the elastomer itself forms the valve element.

20. A surgical access device in accordance with claim 18, wherein the entire access device is made of an elastomer, preferably from a thermoplastic elastomer or an elastomer foam.

21. A surgical access device in accordance with claim 1, wherein it is formed in one piece manner.

22. A surgical access system comprising a surgical access device for introducing surgical instruments into the body of a patient which comprises a sleeve section defining a longitudinal axis, and also comprising an insertion instrument for inserting the access device into an abdominal wall of the patient in particular, wherein a ratio between a length and an internal diameter of the sleeve section lies in a range of approximately 4:1 to approximately 14:1.

23. A surgical access system in accordance with claim 22, wherein a length of the sleeve section lies in a range of approximately 15 mm to approximately 50 mm, preferably within a range of approximately 25 mm to approximately 40 mm.

24. A surgical access system in accordance with claim 22, wherein the insertion instrument comprises a holding section for at least one surgical access device.

25. A surgical access system in accordance with claim 24, wherein a stop acting in the proximal direction is arranged or formed on the distal side of the holding section.
